(19) United States
(12) Patent Application Publication
(10) Pub. No.: US 2023/0093161 A1
(43) Pub. Date: Mar. 23, 2023

(54) METHODS FOR ENVIRONMENTAL CHITIN QUANTIFICATION

(71) Applicant: ARIZONA BOARD OF REGENTS ON BEHALF OF THE UNIVERSITY OF ARIZONA, Tucson, AZ (US)

(72) Inventor: Michael Daines, Tucson, AZ (US)

(21) Appl. No.: 17/934,700

(22) Filed: Sep. 23, 2022

Related U.S. Application Data
(60) Provisional application No. 63/247,497, filed on Sep. 23, 2021.

(51) Int. Cl. G01N 33/58; C08B 37/00

(52) U.S. Cl. CPC G01N 33/581; C08B 37/0003; G01N 2333/42

(57) ABSTRACT

The present invention features a method for solubilizing chitin in an aqueous solution by dissolving a sample containing chitin in a basic solution and autoclaving the sample. The present invention also features a method for quantifying the amount of chitin in a sample. Chitin binding proteins are used to quantify the amount of chitin using a modified ELISA assay. Results of the assay may be compared to a control containing known amounts of chitin to quantify the chitin in the sample.

### CROSS-REFERENCES TO RELATED APPLICATIONS

This application is a non-provisional and claims benefit of U.S. Provisional Application No. 63/247,497 filed Sep. 23, 2021, the specification of which is incorporated herein in its entirety by reference.

### STATEMENT REGARDING FEDERALLY SPONSORED RESEARCH OR DEVELOPMENT

This invention was made with government support under Grant No. R01 Al083403 awarded by National Institutes of Health. The government has certain rights in the invention.

### FIELD OF THE INVENTION

The features methods for quantifying chitin in environmental samples.

### BACKGROUND OF THE INVENTION

Asthma is a chronic inflammatory disease that affects over 35 million people in the United States, and is a leading cause of hospitalization in children. While asthma-causing allergens like fungi, mites, and insects represent biologically diverse organisms, they have some biological similarities that may underlie their potential to cause asthma. One of these shared biological features is the production of environmental proinflammatory microbial associated molecular patterns (MAMP; e.g., chitin). MAMPs (e.g., chitin) impact lung function through the innate immune system despite not being considered as allergens.

Chitin, a modified polysaccharide, is among the most common biopolymers in the world. It is contained in the cell walls of fungi, the exoskeletons of insects, dust mites, and other arthropods, and it is present wherever these organisms are. Thus, the presence of chitin can correlate with environmental exposure to these organisms as a marker of environmental health as well as agricultural contamination with fungal or anthropoid agents. Chitin has been shown to directly activate immune responses, and exposure to it may have a role in the development of asthma. Recent studies have shown that chitin, along with endotoxins and glucans, activate the innate immune system by triggering an allergic response in organisms that are exposed to them

Despite chitin's widespread distribution and clinical relevance, studies of chitin have been restricted by its relative insolubility in common aqueous and organic solvents. Thus, there is an urgent need to develop methods to quantify chitin in environmental samples.

### BRIEF SUMMARY OF THE INVENTION

It is an objective of the present invention to provide methods that allow for the detection and quantification of environmental chitin, as specified in the independent claims. Embodiments of the invention are given in the dependent claims. Embodiments of the present invention can be freely combined with each other if they are not mutually exclusive.

Because humans spend 90% of their time indoors, the present invention seeks to help improve indoor air quality (IAQ) in regards to chitin exposure. Chitin is a non-protein polymer found in the exoskeleton of invertebrates. Exposure to medium size chitin micro-particles (CMPs) has been shown to activate inflammatory immune responses. Additionally, there is a high prevalence of asthma in people working with chitinous substances, supporting a hypothesis that chitin may play a role in asthma development. Thus, in order to associate chitin exposure with lung diseases (e.g., asthma), the present invention provides a novel assay to quantify chitin using wheat germ agglutinin (WGA), a lectin that binds specifically to chitin, to quantify chitin in household dust samples.

Determining chitin levels in dust in homes and commercial buildings may be useful to determine the amount of exposure that individuals and employees have to chitin in their home and work environments. Under the hygiene hypothesis, a dust-sample assay of chitin levels may be useful in assessing the risk of chitin exposure as well as helping in evaluating the benefits of chitin remediation in commercial and residential locations.

Thus, the present invention feature assays for the quantification of chitin in environmental samples. The development of a quantitative assay for chitin is complicated because it is a modified polysaccharide and not a protein. This makes it difficult to make antibodies that will specifically recognize it. Chitin is also poorly soluble in water and many standard solvents, which makes developing adequate standards challenging.

In some embodiments, the present invention may feature a method of processing an environmental sample comprising chitin. In other embodiments, the present invention features a method for converting chitin in an environmental sample to chitosan. The methods may comprise combining an environmental sample comprising chitin with an alkaline solution and heating the alkaline solution and the environmental sample for a period of time. In some embodiments, altering the pH (e.g., by adding the alkaline solution to the environmental sample) and heating the environmental sample converts chitin to chitosan.

In other embodiments, the present invention may also feature a method of quantifying chitin in an environmental sample. The method may comprise converting chitin in the environmental sample to chitosan to form a chitosan sample. The method of converting chitin in an environmental sample to chitosan may comprise combining the environmental sample with an alkaline solution and heating the alkaline solution and the environmental sample for a period of time. In some embodiments, altering the pH (e.g., by adding the alkaline solution to the environmental sample) and heating the environmental sample converts chitin to chitosan and thus creating a chitosan sample. The method of quantifying chitin in an environmental samples may further comprise adding the chitosan sample to a substrate comprising a first chitin binding agent disposed thereon and incubating the chitosan mixture and chitin binding agent for a period of time, washing the substrate, adding a second chitin binding agent to the substrate and measuring an output produced by the reported (e.g., absorbance of the second chitin binding agent bound to the chitosan). In some embodiments, the second chitin binding agent is conjugated to a reporter. In some embodiments, the chitosan is quantified by comparing the measured output (e.g., absorbance) to a standardized curve.

Furthermore, the methods described herein can be used in a plate-based ELISA system for rapid assessment of multiple samples. The method of the present invention was developed in a standard 96 well plate, allowing for multiple samples to be run simultaneously. Additionally, a standard curve of commercially available chitosan is used to precisely quantify the chitin in the environmental samples. Other tests for the presence of chitin are qualitative and can essentially only detect its presence. This is not very useful because there are many sources of chitin and little is known of what limits of exposure may be important.

In further embodiments, the present invention may further feature a kit for quantifying chitin in an environmental sample. The kit may comprise a substrate comprising a first chitin binding agent disposed thereon and second chitin binding agent is conjugated to reporter.

In some embodiments, the present invention may also feature methods for construction (i.e., manufacturing) a substrate comprising a first chitin binding agent disposed thereon. The method may comprise adding a first chitin binding agent to a substrate and incubating for a period of time, washing the substrate, blocking the substrate (to prevent non-specific binding) for a period of time. In some embodiments, the method further comprises sealing and storing the substrate (i.e., the substrate comprising a first chitin binding agent disposed thereon). The substrate may be washed before a chitosan sample and/or chitosan controls are added to the substrate for quantification.

One of the unique and inventive technical features of the present invention is the conversion of chitin into chitosan in an environmental sample by heating and altering the pH of the environmental sample. Without wishing to limit the invention to any theory or mechanism, it is believed that the technical feature of the present invention advantageously allows for the precise and direct quantification of chitin in environmental samples. Making the chitin soluble and able to be dissolved in appropriate solvents for biological analysis is important to the present invention, because chitin, in its insoluble state, would not be able to be assessed and would precipitate out of solution. None of the presently known prior references or work has the unique inventive technical feature of the present invention.

Furthermore, the prior references teach away from the present invention. For example, previous attempts to measure chitin were qualitative. Prior references have tried to examine insoluble chitin which is resistant to most standard analytic techniques. By converting to chitosan the present invention is able to solubilize the converted chitosan in solvents compatible with ELISA techniques, for use in a capture/detect system for precise quantification.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a flow chart showing the sequence of steps for quantifying the amount of chitin in an environmental sample using the methods described herein.

FIG. 2 shows a graph of mean chitin levels vs. the response to the following question: Have you seen any cockroaches during the day in your home?

FIG. 3 shows a graph of mean chitin levels vs. the response to the following question: Have you seen any cockroaches in your home in the last 12 months (Yes/No)?

FIG. 4 shows a histogram depicting the distribution of endotoxin levels in 378 dust samples.

FIG. 5 shows a histogram depicting the distribution of chitin levels in 379 dust samples.

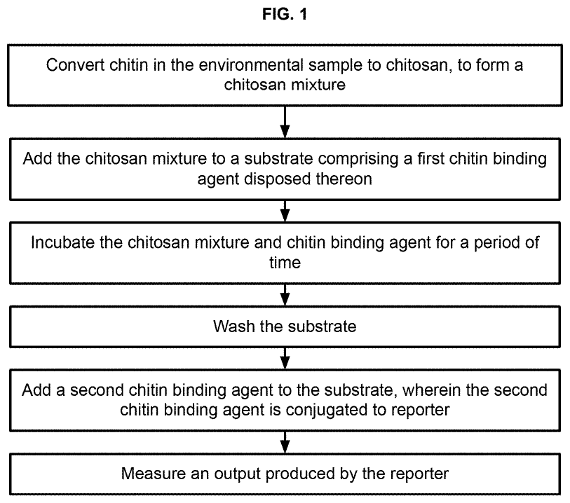

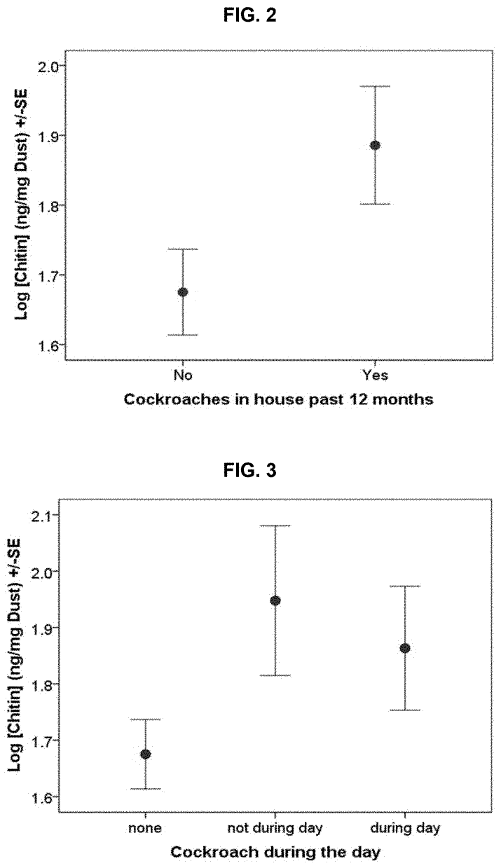

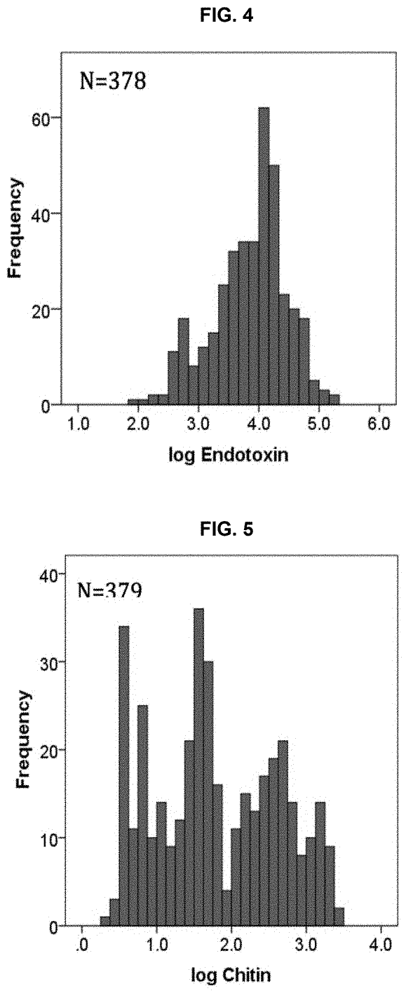

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

The present invention relates to the quantification of chitin from environmental samples. In some embodiments, the process comprises converting chitin to chitosan, then binding the chitosan with wheat germ agglutinin (WGA). Researchers can then determine chitosan concentrations in the environmental samples (e.g., dust) at levels below 50 ng.

Chitin refers to a long-chain polymer of N-acetylglucosamine (i.e., an amide derivative of glucose), and is the second most abundant polysaccharide in nature (behind only cellulose). Chitins from all sources are structurally similar, and they are insoluble in water and relatively resistant to degradation. It is a primary component of cell walls in fungi, and the exoskeletons of arthropods such as crustaceans and insects and can be found anywhere these chitin sources are present, including environmental dust and agricultural samples. Thus, accurately measuring chitin in environmental samples would indicate the presence of chitin producing organisms that may have a role in environmental associated illnesses like allergy or asthma or crop contamination with insects or fungi.

Referring now to the figures, the present invention features methods for converting chitin in an environmental sample to chitosan, as well as methods, kits, and compositions for quantification of chitin in an environmental sample.

The present invention may feature methods of processing an environmental sample comprising chitin. The method may comprise combining an environmental sample comprising chitin with an alkaline solution, and heating the alkaline solution and the environmental sample for a period of time. In some embodiments, altering the pH (e.g., by adding the alkaline solution to the environmental sample) and heating the environmental sample converts chitin to chitosan (i.e., a chitosan sample).

The present invention may further feature methods for converting chitin in an environmental sample to chitosan. The method may comprise combining an environmental sample comprising chitin with an alkaline solution, and heating the alkaline solution and the environmental sample for a period of time. In some embodiments, altering the pH (e.g., by adding the alkaline solution to the environmental sample) and heating the environmental sample converts chitin to chitosan. Converting chitin in an environmental sample to chitosan, allows for the formation of a chitosan sample.

Without wishing to limit the present invention to any theory or mechanism, the combination of the heat and the basic pH of the solution (i.e., the alkaline solution) converts chitin into chitosan.

In some embodiments, the environmental sample may comprise water, soil, dust, airborne particulates, or a combination thereof. In other embodiments, the environmental sample comprises an agricultural sample. Chitin from fungi or arthropods may be present in an environmental sample used in methods described herein.

In some embodiments, the alkaline solution comprises a strong base (i.e., a fully ionic base that is completely dissociated in an aqueous solution). Non-limiting examples of strong bases may include but are not limited to lithium hydroxide (LiOH), sodium hydroxide (NaOH), potassium hydroxide (KOH), calcium hydroxide (Ca(OH)₂), rubidium hydroxide (RbOH), strontium hydroxide (Sr(OH)₂), cesium hydroxide (CsOH), or barium hydroxide (Ba(OH)₂). In certain embodiments, the alkaline solution comprises sodium hydroxide.

In some embodiments, the alkaline solution may contain about 20 to 60% w/v, or about 20 to 50% w/v, or about 20 to 40% w/v, or about 20 to 30% w/v, or about 30 to 60% w/v, or about 30 to 50% w/v, or about 30 to 40% w/v, or about 40 to 60% w/v, or about 40 to 50% w/v, or about 50 to 60% w/v of a strong base. In other embodiments, the alkaline solution may contain about 20% w/v, 30% w/v, 40% w/v, 50% w/v, or 60% w/v of a strong base.

As a non-limiting example, the alkaline solution may contain about 30 to 50% w/v sodium hydroxide. In some embodiments, the alkaline solution may contain about 20 to 60% w/v sodium hydroxide, or about 20 to 50% w/v sodium hydroxide, or about 20 to 40% w/v sodium hydroxide, or about 20 to 30% w/v sodium hydroxide, or about 30 to 60% w/v sodium hydroxide, or about 30 to 50% w/v sodium hydroxide, or about 30 to 40% w/v sodium hydroxide, or about 40 to 60% w/v sodium hydroxide, or about 40 to 50% w/v sodium hydroxide, or about 50 to 60% w/v sodium hydroxide. In other embodiments, the alkaline solution may contain about 20% w/v, 30% w/v, 40% w/v, 50% w/v, or 60% w/v sodium hydroxide.

In some embodiments, the alkaline solution and the environmental sample may be heated to about 90 to 160° C., or about 90 to 150° C., or about 90 to 140° C., or about 90 to 130° C., or about 90 to 120° C., or about 90 to 110° C., or about 90 to 100° C., or about 90 to 95° C., or about 95 to 160° C., or about 95 to 150° C., or about 95 to 140° C., or about 95 to 130° C., or about 95 to 120° C., or about 95 to 110° C., or about 95 to 100° C., or about 100 to 160° C., or about 100 to 150° C., or about 100 to 140° C., or about 100 to 130° C., or about 100 to 120° C., or about 100 to 110° C., or about 110 to 160° C., or about 110 to 150° C., or about 110 to 140° C., or about 110 to 130° C., or about 110 to 120° C., or about 120 to 160° C., or about 120 to 150° C., or about 120 to 140° C., or about 120 to 130° C., or about 130 to 160° C., or about 130 to 150° C., or about 130 to 140° C., or about 140 to 160° C., or about 140 to 150° C., or about 150 to 160° C.

In some embodiments, the alkaline solution and the environmental sample are heated using an incubator. In other embodiments, the alkaline solution and the environmental sample are heated using an autoclave.

In some embodiments, heating the alkaline solution and the environmental sample comprises autoclaving the alkaline solution and the environmental sample. In some embodiments, the alkaline solution and the environmental sample may be autoclaved at about 160° C. In other embodiments, the alkaline solution and the environmental sample may be autoclaved at about 90 to 160° C., or about 90 to 150° C., or about 90 to 140° C., or about 90 to 130° C., or about 90 to 120° C., or about 90 to 110° C., or about 90 to 100° C., or about 90 to 95° C., or about 95 to 160° C., or about 95 to 150° C., or about 95 to 140° C., or about 95 to 130° C., or about 95 to 120° C., or about 95 to 110° C., or about 95 to 100° C., or about 100 to 160° C., or about 100 to 150° C., or about 100 to 140° C., or about 100 to 130° C., or about 100 to 120° C., or about 100 to 110° C., or about 110 to 160° C., or about 110 to 150° C., or about 110 to 140° C., or about 110 to 130° C., or about 110 to 120° C., or about 120 to 160° C., or about 120 to 150° C., or about 120 to 140° C., or about 120 to 130° C., or about 130 to 160° C., or about 130 to 150° C., or about 130 to 140° C., or about 140 to 160° C., or about 140 to 150° C., or about 150 to 160° C.

Without wishing to limit the present invention to any theories or mechanisms it is believed that methods described herein using temperature less than 100° C. may be heated using an incubator, whereas methods described herein using temperatures greater than 100° C. may need to be heated using an autoclave. The use of an autoclave allows for a higher temperature and higher pressure to be used and thus may prevent the sample from boiling away.

In certain embodiments, the alkaline solution and the environmental sample are heated for about an hour. In some embodiments, the alkaline solution and the environmental sample are heated for about 0.5 to 5.0 hours, or about 0.5 to 4.5 hours, or about 0.5 to 4.0 hours, or about 0.5 to 3.5 hours, or about 0.5 to 3.0 hours, or about 0.5 to 2.5 hours, or about 0.5 to 2.0 hours, or about 0.5 to 1.5 hours, or about 0.5 to 1.0 hours, or about 1.0 to 5.0 hours, or about 1.0 to 4.5 hours, or about 1.0 to 4.0 hours, or about 1.0 to 3.5 hours, or about 1.0 to 3.0 hours, or about 1.0 to 2.5 hours, or about 1.0 to 2.0 hours, or about 1.0 to 1.5 hours, or about 1.5 to 5.0 hours, or about 1.5 to 4.5 hours, or about 1.5 to 4.0 hours, or about 1.5 to 3.5 hours, or about 1.5 to 3.0 hours, or about 1.5 to 2.5 hours, or about 1.5 to 2.0 hours, or about 2.0 to 5.0 hours, or about 2.0 to 4.5 hours, or about 2.0 to 4.0 hours, or about 2.0 to 3.5 hours, or about 2.0 to 3.0 hours, or about 2.0 to 2.5 hours, or about 2.5 to 5.0 hours, or about 2.5 to 4.5 hours, or about 2.5 to 4.0 hours, or about 2.5 to 3.5 hours, or about 2.5 to 3.0 hours, or about 3.0 to 5.0 hours, or about 3.0 to 4.5 hours, or about 3.0 to 4.0 hours, or about 3.0 to 3.5 hours, or about 3.5 to 5.0 hours, or about 3.5 to 4.5 hours, or about 3.5 to 4.0 hours, or about 4.0 to 5.0 hours, or about 4.0 to 4.5 hours, or about 4.5 to 5.0 hours. In other embodiments, the alkaline solution and the environmental sample are heated for more than 5 hours.

In some embodiments, the method may further comprise isolating the chitosan. In one example, the isolation of chitosan may be performed by extracting the chitosan from the solution after autoclaving, neutralizing the solution with sulfuric acid, then dissolving the chitosan in acetic acid (0.2M) to further quantify the amount of chitosan in solution.

In some embodiments, the methods described herein may further comprise neutralizing the alkaline solution and the environmental sample mixture subsequent to heating the mixture. The mixture (e.g., comprising the alkaline solution and the environmental sample) may be neutralized with an acidic solution (e.g., a solution comprising sulfuric acid). In some embodiments, the acidic solution comprises a strong acid (i.e., a fully ionic acid that is completely dissociated in an aqueous solution). Non-limiting examples of strong acids that may be used include but are not limited to chloric acid, hydrobromic acid, hydrochloric acid, hydroiodic acid, nitric acid, perchloric acid, and sulfuric acid. To neutralize the mixture (e.g., comprising the alkaline solution and the environmental sample) an acidic solution may be added to the mixture until the final solution reaches a pH around 7 (i.e., a pH of 6.5, 6.6, 6.7, 6.8, 6.9, 7.1, 7.2, 7.3, 7.4, 7.5, ect . . . ).

In some embodiments, the chitosan is dissolved in an acidic solution comprising a weak acid. Non-limiting examples of weak acids include but are not limited to, acetic acid, or citric acid. In some embodiments, the chitosan is dissolved in a weak acid with similar strength to acetic acid. In other embodiments, the chitosan may be dissolved in any solution that is compatible with the method of quantification, such as the methods described herein.

The present invention may also feature a method of quantifying chitin in an environmental sample. The method may comprise converting chitin in the environmental sample to chitosan to form a chitosan sample. The method of converting chitin in an environmental sample to chitosan may comprise combining the environmental sample with an alkaline solution and heating the alkaline solution and the environmental sample for a period of time. In some embodiments, altering the pH (e.g., by adding the alkaline solution to the environmental sample) and heating the environmental sample converts chitin to chitosan. This process (i.e., converting chitin to chitosan) allows for the formation of a chitosan sample. The method of quantifying chitin in a environmental samples may further comprise adding the chitosan sample to a substrate comprising a first chitin binding agent disposed thereon and incubating the chitosan sample and chitin binding agent for a period of time, washing the substrate (e.g., to remove any unbound substances), adding a second chitin binding agent to the substrate and measuring an output produced by the reporter (e.g., measuring absorbance). In some embodiments, the second chitin binding agent is conjugated to a reporter. In some embodiments, the chitosan is quantified by comparing the measured output to a standardized curve.

In some embodiments, the chitosan sample and chitin binding agent are incubated for about 1 to 24 hours, or about 1 to 20 hours, or about 1 to 16 hours, or about 1 to 12 hours, or about 1 to 8 hours, or about 1 to 4 hours, or about 4 to 24 hours, or about 4 to 20 hours, or about 4 to 16 hours, or about 4 to 12 hours, or about 4 to 8 hours, or about 8 to 24 hours, or about 8 to 20 hours, or about 8 to 16 hours, or about 8 to 12 hours, or about 16 to 24 hours, or about 16 to 20 hours, or about 20 to 24 hours. In other embodiments, the chitosan mixture and chitin binding agent are incubated for greater than 24 hours.

Without wishing to limit the present invention to any theory or mechanism it is believed that the sensitivity of the methods described (e.g., methods of quantifying chitin in an environmental sample) increases the longer the chitosan mixture and chitin binding agent are incubated.

In some embodiments, washing the substrate removes any unbound substances and/or non-specific substances (e.g., unbound chitosan). As used herein, “unbound substances,” may refer to any components from the environmental sample and/or the chitosan sample that may inhibit methods described herein. In some embodiments, washing the substrate allows for only the bound chitosan (i.e., chitosan bound to the first chitin binding agent) to remain on the substrate such that chitosan (i.e., chitin) is able to be detected by the methods of the present invention.

In some embodiments, the substrate is washed with a phosphate buffered saline (PBS), e.g., to remove any unbound substances (e.g., unbound chitosan). In some embodiments, the substrate is washed with a phosphate buffered saline (PBS) comprising a detergent (e.g., tween-20) to remove any unbound substances. In other embodiments, the substrate is washed with a Hank's balanced salt solution (HBSS) to remove unbound substances. In some embodiments, the substrate is washed with a Hank's balanced salt solution (HBSS) comprising a detergent (e.g., tween-20) to remove unbound substances. Any balanced salt solution may be used in accordance with the methods described herein to wash the substrate and remove any unbound substances on the substrate. In some embodiments, the balanced salt solution may comprise a non-ionic detergent.

In some embodiments, the first chitin binding agent and/or second chitin binding agent is a chitin-binding lectin or a chitin binding antibody. In some embodiments, the chitin-binding lectin is wheat germ agglutinin. In other embodiments, the first chitin binding agent and/or second chitin binding agent is wheat germ agglutinin.

In some embodiments, the second chitin binding agent is conjugated to a reporter. In some embodiments, the reporter is a fluorescent reporter. In other embodiments, the reporter is an enzymatic reporter (e.g., horseradish peroxidase or alkaline phosphatase, or beta-galactosidase). In further embodiments, the reporter is a radioisotope reporter. In some embodiments, the second chitin binding agent is conjugated to an enzymatic reporter (e.g., the second chitin binding agent is conjugated to horseradish peroxidase). In other embodiments, the second chitin binding agent is conjugated to a fluorescent reporter. In further embodiments, the second chitin binding agent is conjugated to a radioisotope reporter. In certain embodiments, the wheat germ agglutinin is conjugated to a reporter (e.g., an enzymatic reporter or a fluorescent reporter). In other embodiments, the wheat germ agglutinin is conjugated to horseradish peroxidase. Any protein that binds to chitin may be used in the methods described in the present invention.

In some embodiments, the output produced by the reporter is an absorbance (e.g., in methods as described herein using an enzymatic reporter). In other embodiments, the output produced by the reporter is a particular wavelength of light (e.g., an emission of light from a reporter; e.g., in methods as described herein using a fluorescent reporter). In further embodiments, the output produced by the reporter is radiation (e.g., radiation emitted from a reporter; e.g., in methods as described herein using a radioisotope reporter).

In some embodiments, the methods described herein further comprise adding a reagent to the substrate subsequent to adding a second chitin binding agent. The reagents described herein may activate the reporter. For example, when hydrogen peroxide (i.e., a reagent) is added to a second chitin binding agent conjugated to horseradish peroxidase along with a substrate, the horseradish peroxidase enzyme is activated and oxidizes the substrate and to produce a characteristic color change that is detectable by spectrophotometric methods. Non-limiting example of horseradish peroxidase substrates include but are not limited to TMB (3,3′,5,5′-tetramethylbenzidine), ABTS (2,2′-Azinobis [3-ethylbenzothiazoline-6-sulfonic acid]-diammonium salt), OPD (o-phenylenediamine dihydrochloride), or PNPP (p-Nitrophenyl Phosphate). In aforementioned the embodiment, i.e., methods using an enzymatic reporter, the methods described herein may further comprise stopping (i.e., neutralizing) the reaction between the enzymatic reporter and the substrate by adding an acidic solution. In some embodiments, the acidic solution comprises a strong acid. In one embodiment, the reaction (i.e., the enzymatic reporter and the substrate) may be stopped using an acidic solution comprising H₂SO₄ (e.g., 1M H₂SO₄). In certain embodiments, the reaction (i.e., the enzymatic reporter and the substrate) may be stopped using an acidic solution comprising HCl (e.g., 1M HCl). Non-limiting examples of strong acids that may be used to neutralize the reaction between the enzymatic reporter and the substrate include but are not limited to chloric acid, hydrobromic acid, hydrochloric acid, hydroiodic acid, nitric acid, perchloric acid, and sulfuric acid.

Alternatively, light may be used as a reagent. As such, In some embodiments, the methods described herein further comprise exposing the second chitin binding agent conjugated to a fluorescent reporter to a reagent (i.e., light). For example, if the second chitin binding agent is conjugated to a fluorescent reporter, the fluorescent reporter may be detected by exposing the substrate to light (i.e., a reagent) at a particular wavelength.

In another embodiment, the methods described herein may not need a reagent. For example, if the second chitin binding agent is conjugated to a radioisotope reporter, the radiation emitted from the reporter (i.e., the radioisotope reporter) may be directly measured from the sample.

In some embodiments, the method described herein may further comprise washing unbound second chitin binding agents from the substrate (e.g., before adding a reagent or before an output is measured). In some embodiments, the substrate is washed with a phosphate buffered saline (PBS) to remove unbound second chitin binding agents. In some embodiments, the substrate is washed with a phosphate buffered saline (PBS) comprising a detergent (e.g., tween-20) to remove unbound second chitin binding agents. In other embodiments, the substrate is washed with a Hank's balanced salt solution (HBSS) to remove unbound second chitin binding agents. In some embodiments, the substrate is washed with a Hank's balanced salt solution (HBSS) comprising a detergent (e.g., tween-20) to remove unbound second chitin binding agents. Any balance salt solution may be used in accordance with the methods described herein to wash (i.e., remove) unbound second chitin binding agents. In some embodiments, the balanced salt solution may comprise a non-ionic detergent.

In some embodiments, the absorbance is measured at about 400 nm to 650 nm. In some embodiments, the absorbance is measured at about 620 nm, or about 650 nm, or about 652 nm. In other embodiments, the absorbance is measured at about 400 nm, or about 405 nm, or about 410 nm, or about 420 nm, or about 450 nm, or about 490 nm.

In some embodiments, the present invention also features a method for quantifying an amount of chitin in an environmental sample. The method may comprise: a) converting chitin in the environmental sample into chitosan to form a sample comprising chitosan (i.e., a chitosan sample); b) adding the chitosan sample to a substrate comprising first chitin binding agent disposed thereon; c) washing the substrate (e.g, to remove any unbound substances); d) adding a second chitin binding agent conjugated to a reporter; e) washing the substrate to remove any of the unbound second chitin binding agent; f) adding a reagent and incubating for a period of time; g) stopping (e.g., neutralizing) the reaction between the reagent and the report; and h) measuring an absorbance of the sample comprising chitosan. This method may be performed with a known amount of chitin, a control, to produce a standard curve.

In other embodiments, the present invention may feature a method of quantifying chitin in an environmental sample. The method may comprise converting chitin in the environmental sample to chitosan, to form a chitosan sample using methods described herein. The method of quantifying chitin in a environmental sample may further comprise adding the chitosan mixture to a substrate comprising a first chitin binding agent disposed thereon, incubating the chitosan mixture and chitin binding agent for a period of time, washing the substrate to (e.g., to remove any unbound substances), and adding a second chitin binding agent to the substrate. In some embodiments, the second chitin binding agent is conjugated to a reporter. In some embodiments, the method of quantifying chitin in a environmental sample may further comprise exposing the second chitin binding agent conjugated to a fluorescent reporter to light (e.g., exposing the second chitin binding agent conjugated to a fluorescent reporter to a particular wavelength of light) and measuring the wavelength emitted from the fluorescent reporter. In some embodiments, the chitin in the environmental sample is quantified by comparing the wavelength emitted from the chitosan sample to a standardized curve. In other embodiments, the method of quantifying chitin in an environmental sample may further comprise measuring radiation emitted from the radioisotope reporter (i.e., a second chitin binding agent conjugated to a radioisotope reporter). In some embodiments, the chitin in the environmental sample is quantified by comparing the measured radiation from the chitosan sample to a standardized curve.

Certain embodiments of the methods described herein may include first converting chitin from an environmental sample into a sample comprising chitosan, using application of heat (e.g., from an autoclave) and an alkaline solution (e.g., alkaline solution comprising sodium hydroxide). The chitosan protein is soluble and more able to be detected by the assay. The samples comprising chitosan are placed on a substrate comprising wheat gluten agglutinin disposed thereon. The substrate is washed to remove non-specific substances (e.g., unbound chitosan), the plate is treated with a detection molecule (e.g., a second chitin binding agent conjugated to a reporter) and washed to capture the proteins. Researchers can then measure the activity of the chitosan proteins and compare them to known chitosan concentrations to determine chitosan concentrations in the environmental sample at levels below 50 ng.

In some embodiments, the method for quantifying an amount of chitin in an environmental sample may be performed in a 96 well plate. In one embodiment, the chitin is converted into chitosan using an alkaline solution (e.g., a base) and an autoclave. In further embodiments, the substrate containing the chitosan is incubated for a period of time before adding the second chitin binding agent conjugated to a reporter. The period of time may be up to 24 hours.

The present invention may further feature a kit for quantifying chitin in an environmental sample. The kit may comprise a substrate comprising a first chitin binding agent disposed thereon and second chitin binding agent is conjugated to reporter.

In some embodiments, the kit may further comprise control (e.g., a control sample). The control comprises a known amount of chitin. A standard curve may be created using the control. One of ordinary skill in the art would understand how to create a standard curve. However, briefly, the known amount (e.g., concentration) of chitin may be used in a serial dilution (i.e., a process of taking a sample (e.g., a control sample) and diluting it through a series of standard volumes of sterile diluent). This serial diluted chitin sample may then be processed via the methods described herein and used to create a standard curve. In some embodiments, the chitin in the environmental sample may be quantified by comparing the measure absorbance to a standardized curve.

In some embodiments, the present invention also features a kit for quantifying an amount of chitin in an environmental sample. The kit may comprise a substrate comprising a first chitin binding agent disposed thereon, a second chitin binding agent conjugated to a reporter, a reagent, and a control comprising a known amount of chitin. In one embodiment, the kit may further comprise a solution for stopping the reaction between the reagent and the reporter.

In some embodiments, the kit may further comprise a set of instructions for quantifying the amount of chitin in the sample. The set of instructions may comprise instructions for converting chitin in the environmental sample to chitosan to form a sample comprising chitosan (e.g., using methods described herein). The set of instructions may further comprises instructions for using the kit comprising (a) adding the sample comprising chitosan to the substrate comprise the first chitin binding agent disposed thereon, (b) washing the substrate to remove unbound substances, (c) adding the second chitin binding agent conjugated to a reporter to the substrate, (d) washing the substrate to remove any unbound second chitin binding agent, (e) adding the reagent and incubating for a period of time, (f) neutralizing the reaction between the reagent and the reporter, and (g) measuring an absorbance of the sample comprising chitosan. The set of instructions may comprise instructions for creating a standard curve using the control.

In some embodiments, the amount of chitin may be quantified by comparing the absorbance from the environmental sample (e.g., the sample comprising chitosan) to the absorbance from the control.

The present invention may also feature methods for construction (i.e., manufacturing) a substrate comprising a first chitin binding agent disposed thereon. The method may comprise adding a first chitin binding agent to a substrate and incubating for a period of time, washing the substrate, blocking the substrate (to prevent non-specific binding) for a period of time. In some embodiments, the method further comprises sealing and storing the substrate (i.e., the substrate comprising a first chitin binding agent disposed thereon). The substrate may be washed before a chitosan sample and/or chitosan controls are added to the substrate for quantification.

In some embodiments, the substrate is a 96-well plate.

In some embodiments, the first chitin binding agent and the substrate are incubated for about 16 hours. In other embodiments, the first chitin binding agent and the substrate are incubated for about 10 to 48 hours, or about 10 to 42 hours, or about 10 to 36 hours, or about 10 to 30 hours, or about 10 to 24 hours, or about 10 to 18 hours, or about 10 to 16 hours, or about 10 to 15 hours, or about 15 to 48 hours, or about 15 to 42 hours, or about 15 to 36 hours, or about 15 to 30 hours, or about 15 to 24 hours, or about 15 to 18 hours, or about 15 to 16 hours, or about 16 to 48 hours, or about 16 to 42 hours, or about 16 to 36 hours, or about 16 to 30 hours, or about 16 to 24 hours, or about 16 to 18 hours, or about 18 to 48 hours, or about 18 to 42 hours, or about 18 to 36 hours, or about 18 to 30 hours, or about 18 to 24 hours, or about 24 to 48 hours, or about 24 to 42 hours, or about 24 to 36 hours, or about 10 to 30 hours, or about 30 to 48 hours, or about 30 to 42 hours, or about 30 to 36 hours, or about 36 to 48 hours, or about 36 to 42 hours, or about 42 to 48 hours. In some embodiments, the first chitin binding agent and the substrate are incubated for more than 48 hours.

In some embodiments, the first chitin binding agent and the substrate are incubated at or about room temperature (e.g., about 15° C. to 25° C.). In other embodiments, the first chitin binding agent and the substrate are incubated at about 10° C. to 30° C., or about 10° C. to 25° C., or about 10° C. to 20° C., or about 10° C. to 15° C., or about 15° C. to 30° C., or about 15° C. to 25° C., or about 15° C. to 20° C., or about 20° C. to 30° C., or about 20° C. to 25° C., or about 25° C. to 30° C.

In some embodiments, the first chitin binding agent and the substrate are washed with a phosphate buffered saline (PBS) to remove the unbound first chitin binding agent. In some embodiments, the first chitin binding agent and the substrate are washed with a phosphate buffered saline (PBS) comprising a detergent (e.g., tween-20) to remove the unbound first chitin binding agent. In other embodiments, the first chitin binding agent and the substrate are washed with a Hank's balanced salt solution (HBSS) to remove the unbound first chitin binding agent. In some embodiments, the first chitin binding agent and the substrate are washed with a Hank's balanced salt solution (HBSS) comprising a detergent (e.g., tween-20) to remove the unbound first chitin binding agent. Any balance salt solution may be used in accordance with the methods described herein to wash the first chitin binding agent and the substrates. In some embodiments, the balanced salt solution may comprise a non-ionic detergent.

In some embodiments, the substrate is blocked with bovine serum albumin (BSA; e.g., 300 uL of 2% BSA in 50 mM Tris-HCl blocking buffer). The substrate may be blocked with any solution that blocks nonspecific binding, including but not limited to nonfat milk (e.g., a solution comprising 5% nonfat dry milk), whole serum (e.g., a solution comprising 10% whole serum), or fish gelatin (e.g., a solution comprising fish gelatin).

In some embodiments, the substrate comprising the blocking buffer is incubated for about 3 hours. In other embodiments, the substrate comprising the blocking buffer is incubated for about 0.5 to 5 hours, or about 0.5 to 4 hours, or 0.5 to 3 hours, or about 0.5 to 2 hours, or about 0.5 to 1 hour, or about 1 to 5 hours, or about 1 to 4 hours, or about 1 to 3 hours, or about 1 to 2 hours, or about 2 to 5 hours, or about 2 to 4 hours, or about 2 to 3 hours, or about 3 to 5 hours, or about 3 to 4 hours, or about 4 to 5 hours.

In some embodiments, the substrate comprising the blocking buffer is incubated at or about room temperature (e.g., about 15° C. to 25° C.). In other embodiments, the substrate comprising the blocking buffer is incubated at about 10° C. to 30° C., or about 10° C. to 25° C., or about 10° C. to 20° C., or about 10° C. to 15° C., or about 15° C. to 30° C., or about 15° C. to 25° C., or about 15° C. to 20° C., or about 20° C. to 30° C., or about 20° C. to 25° C., or about 25° C. to 30° C.

In some embodiments, the method further comprises sealing the substrate and storing the seal substrate at −20° C. for about 6 months. Without wishing to limit the present invention to any theory or mechanism it is believed that storage of the substrate comprising a first chitin binding agent disposed thereon at about −20° C. for about 6 months will not cause significant loss of the binding activity of the first chitin binding agent.

In some embodiments, the substrate comprising a first chitin binding agent disposed thereon is washed before use in methods described herein.

### Example

The following is a non-limiting example of the present invention. It is to be understood that said example is not intended to limit the present invention in any way. Equivalents or substitutes are within the scope of the present invention.

In order to associate chitin exposure with lung diseases like asthma, the inventors have developed a novel assay using wheat germ agglutinin (WGA), a lectin that binds specifically to chitin, to quantify chitin in household dust samples. Chitin concentrations in 387 household dust samples, kindly provided to us by the Inner-City Anti-IgE Therapy for Asthma (ICATA) clinical trial, were determined using this assay.

### Method of Detection

Creation of a quantitative assay for chitin is complicated by several factors. It is a modified polysaccharide, not a protein, so it is difficult to make antibodies that will specifically recognize it. Chitin is poorly soluble in water and in many standard solvents, thus developing adequate standards is challenging. Chitin was extracted by treating the dust samples with 30-50% w/v sodium hydroxide and autoclaving the samples at high temperature (121° C.) for an hour to convert the water insoluble chitin to water-soluble chitosan. The resulting chitosan was extracted, neutralized with H₂SO₄ and dissolved in 0.2M acetic acid for quantification using a modified ELISA assay.

### Converted Chitin Quantification

Chitosan is a substance that contains an acetylated unit called N-acetyl-D-glucosamine. To quantify the converted chitin product, inventors developed a modified ELISA assay using a lectin known as wheat germ agglutinin that selectively binds to N-acetyl-D-glucosamine. 100 μL of 80 ug/mL WGA (Sigma L-9640) was added to 96 well plates for coating at room temperature for over 16 hours. After incubation, the plate is washed and blocked with 300 μL of 2% bovine serum albumin in a 50 mM Tris-HCl blocking buffer and incubated on a shaker at room temperature for 3 hours. At this step, the plate can be sealed and stored at −20° C. for 6 months without significant loss of WGA-chitin binding activity.

Then, the plate is washed before 100 μL of converted chitin samples and chitosan controls (Sigma 448877) are added to the plate. The plate is placed on the shaker for 15 minutes and then stored at 4° C. overnight. The next day, the plate is washed and 100 uL of 0.5 μg/mL of conjugated WGA (Sigma L-3892) is added to each of the wells. The plate is placed on a shaker for 15 minutes and then washed. 100 μL of Substrate Reagent Pack (R&D Systems) is mixed and added to each well of the plate according to protocol. The plate is then placed on an ELISA plate reader and its absorbance at 620 nm is recorded. 100 μL of 1M H₂SO₄ is used to stop the reaction and absorbance at 410 nm is recorded.

### Results

The chitin assay of the present invention was validated with multiple controls and was determined it has detection limit of approximately 20 ng chitin/mg dust. Inventors are determining the amount of chitin in dust and investigating the impact of chitin exposure on the severity of allergic asthma and response to asthma treatment.

### CONCLUSIONS

Understanding the impact of immune modulating microbial components in asthma is important. Overall, this present invention will contribute to understanding the mechanisms in human asthma. It also has the potential to improve existing techniques in the treatment and prevention of a disease that affects millions of people worldwide.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of processing an environmental sample comprising chitin, the method comprising:
a) combining the environmental sample comprising chitin with a alkaline solution; and
b) heating the alkaline solution and the environmental sample for a period of time;
wherein altering pH and heating the environmental sample converts chitin to chitosan.

2. The method of claim 1, wherein the environmental sample comprises water, soil, dust, airborne particulates or a combination thereof.

3. The method of claim 1, wherein the alkaline solution comprises sodium hydroxide.

4. The method of claim 3, wherein the alkaline solution contains about 30-50% w/v sodium hydroxide.

5. The method of claim 1, wherein heating the alkaline solution and the environmental sample comprises autoclaving the alkaline solution and the environmental sample.

6. The method of claim 5, wherein the alkaline solution and the environmental sample is autoclaved at 160° C.

7. The method of claim 1, wherein the alkaline solution and the environmental sample are heated for 1 hour.

8. The method of claim 1, further comprising extracting and neutralizing the chitosan.

9. The method of claim 8, wherein an acidic solution is used to neutralize the chitosan.

10. The method of claim 9, further comprising dissolving the neutralized chitosan in an acidic solution.

11. A method of quantifying chitin in a environmental sample, the method comprising:
a) converting chitin in the environmental sample to chitosan, to form a chitosan sample;
b) adding the chitosan mixture to a substrate comprising a first chitin binding agent disposed thereon,
c) incubating the chitosan mixture and chitin binding agent for a period of time;
d) washing the substrate;
e) adding a second chitin binding agent to the substrate, wherein the second chitin binding agent is conjugated to a reporter; and
f) measuring an output produced by the reporter;
wherein chitin in the environmental sample is quantified by comparing the measured output of the chitosan sample to a standardized curve.

12. The method of claim 11, wherein converting the chitin in an environmental sample to chitosan comprising:
a) combining the environmental sample with a alkaline solution; and
b) heating the alkaline solution and the environmental sample for a period of time;
wherein altering the pH and heating the environmental sample converts chitin to chitosan.

13. The method of claim 11, wherein washing the substrate removes any unbound substances.

14. The method of claim 11, wherein the chitin binding agent comprises a chitin-binding lectin.

15. The method of claim 11, wherein the chitin binding agent comprises wheat germ agglutinin.

16. The method of claim 11, wherein the reporter is a fluorescent reporter or an enzyme reporter

17. The method of claim 16, wherein the enzyme reporter is horseradish peroxidase.

18. A kit for quantifying chitin in an environmental sample, the kit comprising:
a) a substrate comprising a first chitin binding agent disposed thereon;
b) a second chitin binding agent is conjugated to a reporter.

19. The kit of claim 18, further comprising a control, wherein the control comprises a known amount of chitin.

20. The kit of claim 18, wherein the first chitin binding agent and the second chitin agent comprises a chitin-binding lectin.
